(19) United States
(12) Patent Application Publication
(10) Pub. No.: US 2024/0277936 A1
(43) Pub. Date: Aug. 22, 2024

(54) Feedback mechanism for a medicament delivery device

(71) Applicant: SHL Medical AG, Zug (CH)

(72) Inventor: Anders Boström, Nacka Strand (SE)

(21) Appl. No.: 18/693,303

(22) PCT Filed: Sep. 14, 2022
(86) PCT No.: PCT/EP2022/075576

§ 371 Date: Mar. 19, 2024

(30) Foreign Application Priority Data
Sep. 28, 2021 (EP) 21199483.5

(51) Int. Cl. A61M 5/178

(52) U.S. Cl. CPC A61M 5/178; A61M 2205/581; A61M 2205/582

(57) ABSTRACT

The present disclosure provides a feedback mechanism for a medicament delivery device, comprising: a rotator, and a ring member arranged coaxially with the rotator, wherein the rotator and the ring member are configured to be rotatable relative to each other, wherein one of the rotator and the ring member has a radially resilient feedback structure and the other one of the rotator and the ring member has a first counter feedback structure configured to interact with the feedback structure when one of the rotator and the ring member is rotated relative to the other one of the rotator and the ring member, wherein when the feedback structure interacts with the first counter feedback structure the feedback structure is configured to relax and hit the first counter feedback structure to thereby provide a first feedback sound.

### CROSS-REFERENCE TO RELATED APPLICATIONS

The present application is a U.S. National Phase Application pursuant to 35 U.S.C. § 371 of International Application No. PCT/EP2022/075576 filed Sep. 14, 2022, which claims priority to European Patent Application No. 21199483.50 filed Sep. 28, 2021. The entire disclosure contents of these applications are herewith incorporated by reference into the present application.

### TECHNICAL FIELD

The present disclosure generally relates to medicament delivery devices.

### BACKGROUND

Medicament delivery devices such as autoinjectors may be configured to provide an indication to the user that the medicament administration has started or been finalised. This indication may for example be an audible click.

An example of a medicament delivery device that can provide an audible and/or tactile signal indicating that the medicament has been completely expelled is disclosed in WO2011123024 A1. A distally biased U-bracket is provided around the plunger rod. The U-bracket is released from an axially fixed position relative to the plunger rod when the plunger rod has moved fully in the proximal direction, causing the U-bracket to move in the distal direction and impact a surface. This causes an audible click indicating to the user that the medicament delivery has been completed.

One drawback with the configuration of WO2011123024 A1 is that it may complicate assembly of the medicament delivery device and that it has to be manufactured in metal to be able to withstand the forces that it is subjected to in the assembled state.

### SUMMARY

An object of the present disclosure is thus to provide a feedback mechanism which solves, or at least mitigates, problems of the prior art.

There is hence according to a first aspect of the present disclosure provided a feedback mechanism for a medicament delivery device, comprising: a rotator, and a ring member arranged coaxially with the rotator, wherein the rotator and the ring member are configured to be rotatable relative to each other, wherein one of the rotator and the ring member has a radially resilient feedback structure and the other one of the rotator and the ring member has a first counter feedback structure configured to interact with the feedback structure when one of the rotator and the ring member is rotated relative to the other one of the rotator and the ring member, wherein when the feedback structure interacts with the first counter feedback structure the feedback structure is configured to relax and hit the first counter feedback structure to thereby provide a first feedback sound.

Due to the rotational interaction between the ring member and the rotator no preloading with such force as the axially biased U-bracket of WO2011123024 A1 is required. This may make the assemble process less complex. The ring member may furthermore also be manufactured from a wider range of materials such as a polymer material.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device.

When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component.

Similarly, “radial” or “radially” refer to a direction extending radially relative to the axis, and “rotation”, “rotational” and “rotationally” refer to rotation relative to the axis.

According to one embodiment the feedback structure is an arm.

According to one embodiment the first counter feedback structure comprises a radial first step which when interacting with the feedback structure causes the feedback structure to become radially biased, and a radial first step-back following the first step in the circumferential direction of the feedback mechanism configured to be hit by the feedback structure when the feedback structure is relaxed to provide the first feedback sound.

According to one embodiment initial rotation of the rotator relative to the ring member causes the feedback structure to become radially biased by the first step.

According to one embodiment further relative rotation between the ring member and the rotator causes the feedback structure to relax by the first step-back.

According to one embodiment the rotator is configured to be rotated in a first direction and the ring member is configured to be rotated in a second direction opposite to the first direction.

According to one embodiment the rotator is configured to be rotated first causing interaction of the feedback structure with the first step, and the ring member is configured to be rotated second causing interaction of the feedback structure with the first step-back.

According to one embodiment the one of the rotator and the ring member which has the first counter feedback structure has a second counter feedback structure configured to interact with the feedback structure when the ring member is rotated relative to the rotator, wherein the second counter feedback structure comprises a radial second step following the first step-back in the circumferential direction, which when interacting with the feedback structure causes the feedback structure to become radially biased a second time, and a radial second step-back following the second step in the circumferential direction configured to be hit by the feedback structure when the feedback structure is relaxed a second time to provide a second feedback sound indicative of an end of medicament delivery.

According to one embodiment the rotator is configured to be rotated first causing interaction of the feedback structure with the first step and first step-back, and the ring member is configured to be rotated second causing interaction of the feedback structure with the second step and the second step-back.

One embodiment comprises a plunger rod, wherein the rotator and the ring member are arranged around the plunger rod, the plunger rod being configured to move linearly from a first axial position to a second axial position relative to the rotator and the ring member, wherein the plunger rod has a cam surface and the ring member has a guide surface, the cam surface being configured to interact with the guide surface to cause rotation of the ring member relative to the rotator when the plunger rod is moved from the first axial position towards the second axial position, thereby causing the feedback structure to rotate relative to the first counter feedback structure such that the feedback structure is relaxed.

According to one embodiment the first feedback sound is a first click indicative of a start of medicament delivery or a second click indicative of an end of medicament delivery.

There is according to a second aspect of the present disclosure provided a medicament delivery device comprising a feedback mechanism according to the first aspect.

One embodiment comprises: a housing having a proximal end and a distal end, a plunger rod biased towards the proximal end, arranged in the housing, a rear cap comprising a radially flexible arm, wherein the rotator is configured to bear against the arm such that the arm engages with and holds the plunger rod in a first axial position, and a delivery member cover configured to be moved linearly in the housing from an extended position to a retracted position relative to the housing, causing rotation of the rotator which enables the arm to disengage from the plunger rod to thereby release the plunger rod from the first position.

Generally, all terms used in the claims are to be interpreted according to their ordinary meaning in the technical field, unless explicitly defined otherwise herein. All references to “a/an/the member, apparatus, component, means, etc.” are to be interpreted openly as referring to at least one instance of the member, apparatus, component, means, etc., unless explicitly stated otherwise.

### BRIEF DESCRIPTION OF THE DRAWINGS

The specific embodiments of the inventive concept will now be described, by way of example, with reference to the accompanying drawings, in which:

FIG. 1 is a perspective view of an example of a medicament delivery device;

FIG. 2 shows an exploded view of the medicament delivery device in FIG. 1;

FIG. 3 is a perspective view of an example of a feedback mechanism of the medicament delivery device in FIG. 1;

FIG. 4 is a perspective view of a ring member;

FIG. 5 is a perspective view of a rotator;

FIGS. 6A-6C show the operation of the feedback mechanism in FIG. 3 at various stages of medicament delivery;

FIG. 7 shows a perspective view of another example of a feedback mechanism;

FIGS. 8A-8C show the operation of the feedback mechanism in FIG. 7 at various stages of medicament delivery;

FIG. 9A shows yet another example of a feedback mechanism; and

FIG. 9B shows the feedback mechanism in FIG. 9A in a state before medicament delivery has commenced.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like members throughout the description.

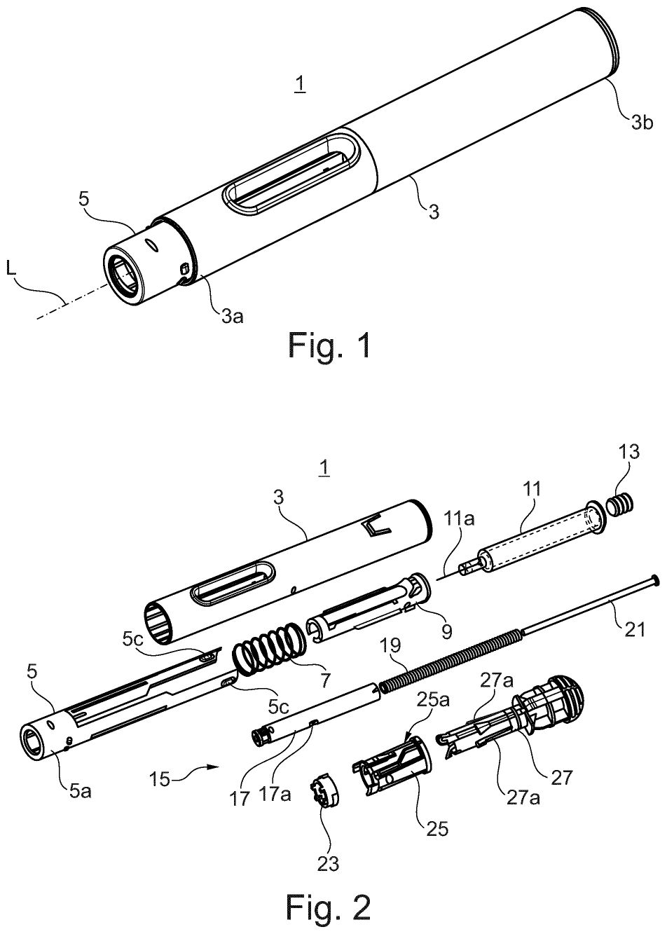

FIG. 1 shows a perspective view of an example of a medicament delivery device 1. In the present example the medicament delivery device 1 is an autoinjector.

The medicament delivery device 1 comprises a housing 3. The housing 3 has a proximal end 3a. The housing 3 has a distal end 3b.

The housing 3 has an elongated shape extending from the proximal end 3a to the distal end 3b. The housing 3 may for example have a tubular shape.

The medicament delivery device 1 comprises a delivery member cover 5. The delivery member cover 5 is arranged in the housing 3. The delivery member cover 5 extends from a proximal opening at the proximal end 3a of the housing 3.

The delivery member cover 5 is configured to be moved axially along a longitudinal axis L of the housing 3. The delivery member cover 5 is configured to be moved from an extended position relative to the housing 3 and the proximal end 3a to a retracted position. The extended position is shown in FIG. 1. The delivery member cover 5 is received further by the housing 3 in the retracted position.

The delivery member cover 5 is rotationally locked relative to the housing 3.

FIG. 2 shows an exploded view of the medicament delivery device 1.

The delivery member cover 5 is elongated. The delivery member cover 5 has a proximal tubular or essentially tubular portion 5a. The delivery member cover 5 has two legs 5b extending from the portion 5a towards the distal end 3b of the housing 3. Each leg 5b has an inner surface provided with a structure 5c, such as a radially inwards extending protrusion.

The exemplified medicant delivery device 1 comprises a first resilient member 7. The first resilient member 7 may be a spring. The first resilient member 7 is configured to bias the delivery member cover 5 towards the extended position.

The medicament delivery device 1 comprises a medicament container holder 9 arranged in the housing 3. The medicament container holder 9 is configured to hold a medicament container 11 such as a syringe. The medicament container 11 has a delivery member 11a such as a needle.

The first resilient member 7 is in the present example configured to bear against an inner distal surface of the portion 5a of the delivery member cover 5 and the medicament container holder 9 to thereby bias the delivery member cover 5 towards the extended position.

The medicament delivery device 1 comprises a stopper 13 configured to be received inside the medicament container 11 and configured to tightly bear against the inner surface of the medicament container 11.

The medicament delivery device 1 further comprises a plunger rod 17, a second resilient member 19, such as a spring, a rod 21, an annular ring member 23, a rotator 25 and a rear cap 27. The rear cap 27 is configured to close the distal end 3b of the housing 3.

The plunger rod 17 is configured to be moved from a first axial position in the housing 3, in which the plunger rod 17 is arranged prior to medicament delivery to a second axial position which is the position that the plunger rod 17 attains when medicament delivery has been performed.

The plunger rod 17 is arranged distally relative to the stopper 13. The plunger rod 17 is configured to move the stopper 13 towards the proximal end of the medicament container 11 when the plunger rod 17 is moved from the first axial position to the second axial position.

Medicament contained in the medicament container 11 is thus pushed through the delivery member 11a.

The second resilient member 19 is configured to be arranged inside the plunger rod 17, which is hollow. The second resilient member 19 is arranged around the rod 21. The plunger rod 17 has an inner radial wall against which the proximal end of the second resilient member 19 rests. The distal end of the second resilient member 19 bears against a radial surface in the rear cap 27. The second resilient member 19 is configured to bias the plunger rod 17 towards the proximal end 3a of the housing 3.

The ring member 23 is arranged coaxially with and configured to be in direct contact with the rotator 25. The ring member 23 is arranged proximally relative to the rotator 25. The ring member 23 could alternatively be arranged distally relative to the rotator 25.

The rotator 25 is arranged around a proximally extending tubular portion of the rear cap 27 arranged inside the housing 3. The proximally extending tubular portion of the rear cap 27 is hollow and the plunger rod 17 is arranged inside the proximally extending tubular portion of the rear cap 27.

The proximally extending tubular portion has radially flexible arms 27a. The plunger rod 17 which is arranged radially inside the proximally extending tubular portion has corresponding recesses 17a configured to engage with a respective one of the radially flexible arms 27a.

The rotator 25, which is arranged radially outside of the proximally extending portion of the rear cap 27 prevents the radially flexible arms 27a to flex radially outwards and thus disengage from the recesses 17a before the delivery member cover 5 has been moved from the extended position to the retracted position.

The rotator 25 has guide structures 25a comprising cam surfaces on its outer surface configured to interact with the structures 5c, such as radially inwards extending protrusions, of the delivery member cover 5.

When the user is to perform medicament delivery, the medicament delivery device 1 is pushed against the site of injection causing the delivery member cover 5 to move from the extended position towards the retracted position. This linear movement causes the rotator 25 to rotate as a result of the interaction between the structures 5c and the guide structures 25a. The rotator 25 is provided with windows or recesses on its inner surface, which when the rotator has been rotated align with a respective one of the radially flexible arms 27a, which as a result can flex radially outwards. The radially flexible arms 27a thus disengage from the recesses 17a and the plunger rod 17 is moved from the first axial position towards the second axial position.

The plunger rod 17 is rotationally locked relative to the rear cap 27. The plunger rod 17 thus moves linearly inside the housing 3 when the plunger rod 17 has been released from the rear cap 27.

The plunger rod 17, the ring member 23, and the rotator 25 form or form part of a feedback mechanism 15.

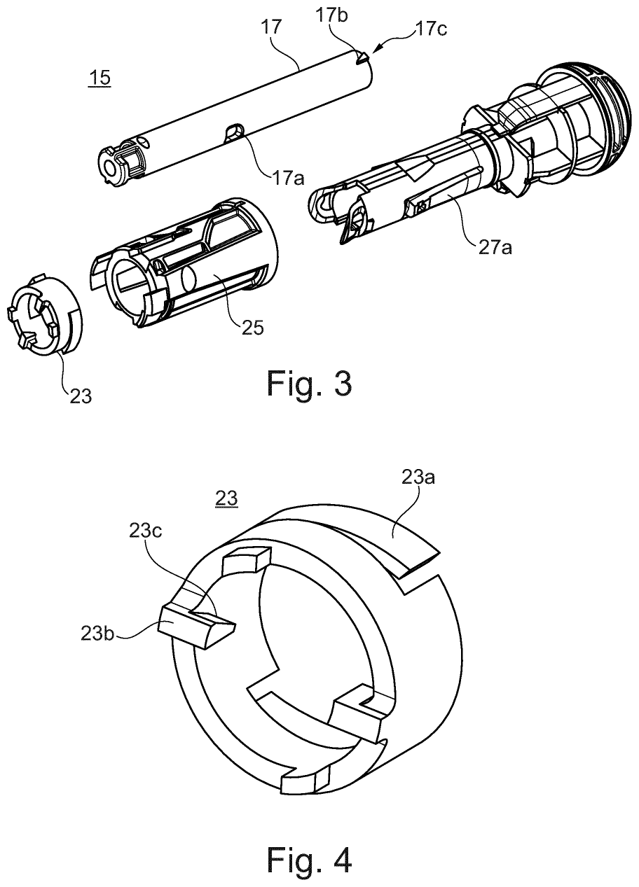

FIG. 3 shows a close-up view of components of the feedback mechanism 15.

The plunger rod 17 has a cam surface 17b. The cam surface 17b is provided on a protrusion 17c extending radially outwards from the outer surface of the plunger rod 17. The cam surface 17b is inclined and defines a plane that intersects a central plane dividing the plunger rod 17 into two halves. The radially outwards extending protrusion 17c may be wedge-shaped. The radially outwards extending protrusion 17c may be provided closer to the distal end of the plunger rod 17 than to its proximal end. The radially outwards extending protrusion 17c may for example be provided at the distal end of the plunger rod 17.

The protrusion 17c is configured to engage with a corresponding axial groove provided on the inner surface of the rear cap 27. The axial groove is arranged inside the proximally extending tubular portion of the rear cap 27. The plunger rod 17 is thus rotationally locked relative to the rear cap 27 by means of the protrusion 17c and the axial groove which receives the protrusion 17c. The rear cap 27 may be rotationally locked relative to the housing 3.

FIG. 4 is a perspective view of the ring member 23. The ring member 23 comprises a radially resilient feedback structure 23a. The feedback structure 23a may be or comprise an arm. The feedback structure 23a extends in the circumferential direction of the ring member 23. The feedback structure 23a extends radially from the outer surface of the ring member 23a when the feedback structure 23a is in a relaxed state. This extension is gradual in the circumferential direction of the ring member 23. The feedback structure 23a can be flexed radially inwards when subjected to a force comprising a radially inwards directed force component.

The exemplified ring member 23 comprises a radially inwards extending structure 23c extending from the inner surface of the annular ring member 23. The radially inwards extending structure 23c has a guide surface 23b. The guide surface 23b is inclined and defines a plane that intersects a central plane dividing the ring member 23 into two halves. The guide surface 23b is configured to interact with the cam surface 17b of the plunger rod 17 when the plunger rod 17 moves towards the second axial position. The ring member 23, which is rotatable relative to the housing 3 will thus be rotated as the plunger rod 17 moves linearly as the plunger rod 17 is about to reach the second axial position.

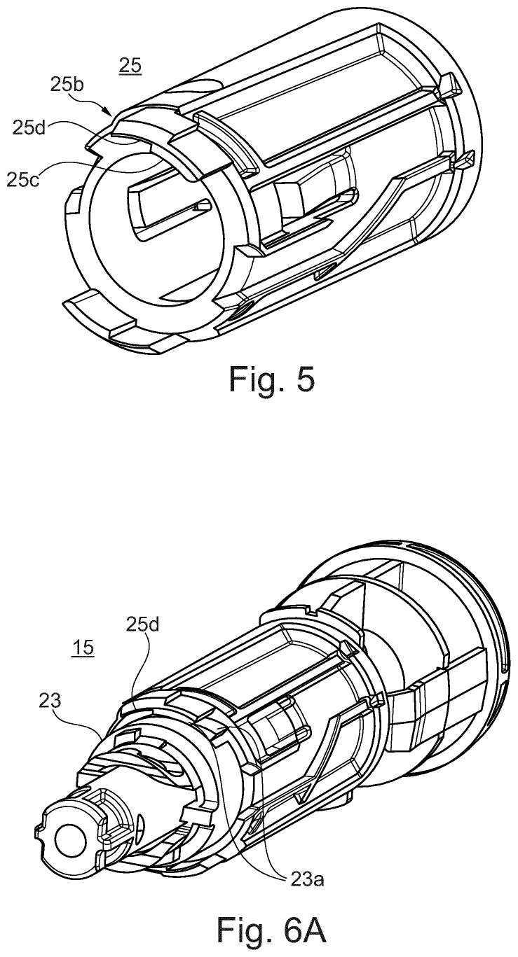

FIG. 5 depicts the rotator 25. The rotator 25 has a first counter feedback structure 25b. The first counter feedback structure 25b is configured to interact with the feedback structure 23a when the rotator 25 and the ring member 23 are rotated relative to each other. The first feedback structure 25b comprises a radial first step 25c and a radial first step-back 25d following the first step 25c in the circumferential direction of the rotator 25. The first step 25c transitions into the first step-back. The first step 25c extends further radially inwards towards the ring member 23 than the first step-back 25d.

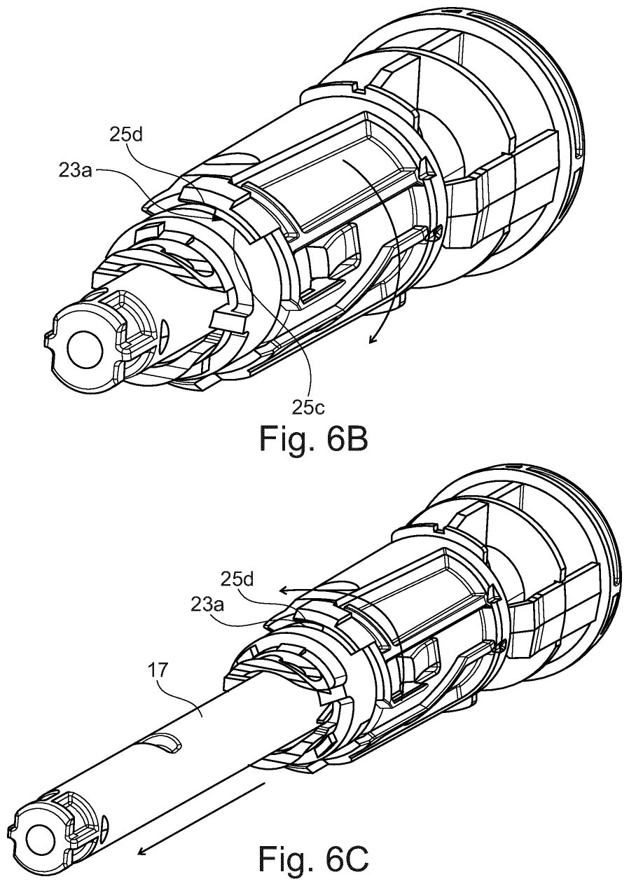

The operation of the feedback mechanism 15 will now be described with reference to FIGS. 6A-6C.

In FIG. 6A, the feedback mechanism 15 is shown before a medicament delivery operation has commenced. The ring member 23 is in an initial rotational position in which the radial first step 25c is arranged offset in the circumferential direction from the feedback structure 23a and the feedback structure 23a is in a relaxed state.

Turning now to FIG. 6B, as the delivery member cover 5 is moved from the extended position towards the retracted position during a medicament delivery operation, the rotator 25 is rotated in a first rotational direction as shown by the arrow. The first counter feedback structure 25b is thus also rotated and the radial first step 25c moves radially over the feedback structure 23a when the rotator 25 is rotated. The feedback structure 23a thus becomes radially biased towards the centre of the ring member 23.

FIG. 6C shows when the medicament delivery operation has ended. As the rotator 25 is rotated the plunger rod 17 is eventually released. The plunger rod 17 is thus moved from the first axial position to the second axial position, which is the final axial position of the plunger rod 17. The cam surface 17b of the plunger rod 17 interacts with the guide surface 23c of the ring member 23 as the plunger rod 17 is moved in the proximal direction, causing the ring member 23 to rotate in a second direction which is opposite to the first direction, as shown by the arrow. The feedback structure 23a is thus moved from underneath the radial first step 25c to align with the radial first step-back 25d which provides space for the feedback structure 23a to relax. The feedback structure 23a thus flexes out and hits the first step-back 25d, causing a first feedback sound. In this case, since the feedback structure 23a hits the first step-back 25d when the plunger rod 17 reaches its second axial position the first feedback sound is indicative of an end of medicament delivery.

The feedback structure and the first counter feedback structure could alternatively change place with each other, with the feedback structure being provided on the rotator and the first counter feedback structure being provided on the ring member.

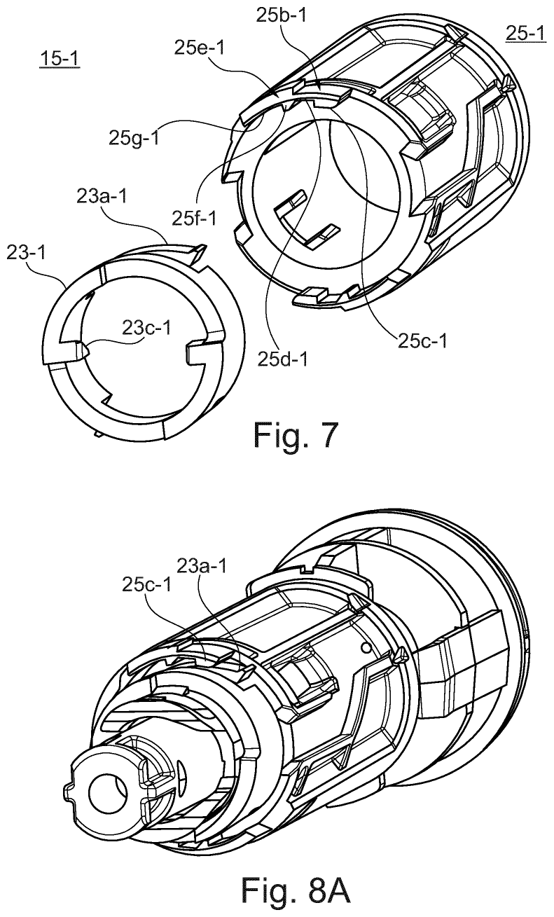

FIG. 7 shows another example of a ring member and a rotator of an alternative feedback mechanism 15-1. In this example, the ring member 23-1 is similar to the ring member 23. The ring member 23-1 comprises a feedback structure 23a-1 similar to the feedback structure 23a.

The rotator 25-1 comprises a first counter feedback structure 25b-1, similar to the first counter feedback structure 25b. The first step 25c-1 however has a shorter extension in the circumferential direction than the first step 25c of the first example.

The rotator 25-1 also comprises a second counter feedback structure 25e-1. The second counter feedback structure 25e-1 comprises a radial second step 25f-1 following the first step-back 25d in the circumferential direction. The second counter feedback structure 25e-1 further comprises a radial second step-back 25g-1 following the second step 25f-1 in the circumferential direction.

The radial second step 25f-1 transitions into the second step-back 25g-1. The second step 25f-1 extends further radially inwards towards the ring member 23-1 than the second step-back 25g-1.

In a circumferential direction of the rotator 25-1 the first step 25c-1 is followed by the first step-back 25d-1, followed by the second step 25f-1, followed by the second step-back 25g-1. The first step 25c-1, the first step-back 25d-1, the second step 25f-1, and the second step-back 25g-1 are arranged one after the other in the circumferential direction of the rotor 25-1.

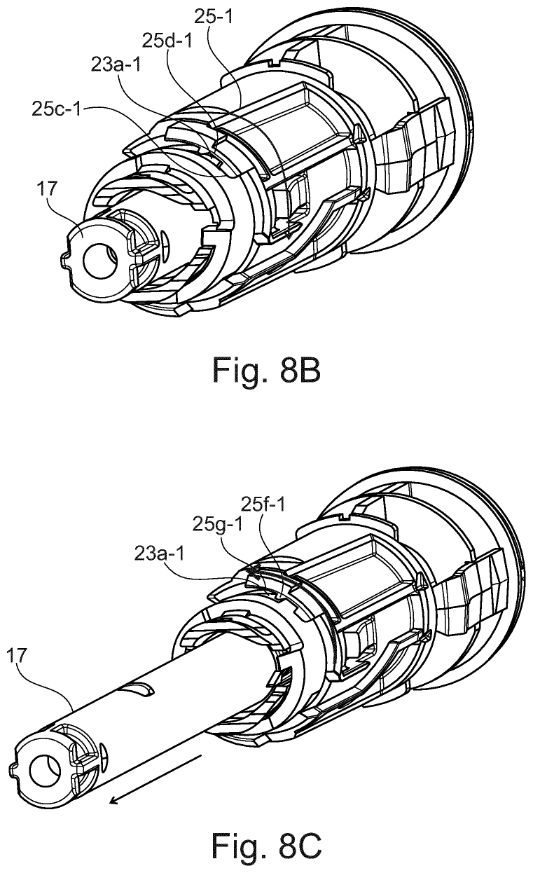

The ring member 23-1 and the rotator 25-1 provide an indication of both start of medicament delivery and end of medicament delivery as will be explained with reference to FIGS. 8A-8C.

FIG. 8A depicts the feedback mechanism 15-1 before a medicament delivery operation has started. The feedback structure 23a-1 is in a relaxed state and in the circumferential direction arranged adjacent to the first step 25c-1.

In FIG. 8B, the rotator 25-1 has been rotated by the linear movement of the delivery member cover 5, causing the release of the plunger rod 17. During this rotational motion of the rotor 25-1, indicated by the arrow, the first step 25c-1 first moves onto the feedback structure 23a-1 causing it to be biased radially inwards, and then moves past the feedback structure 23a-1 due to its circumferential extension being shorter than in the first example. The feedback structure 23a-1 thus relaxes and hits a radially inwards facing surface of the first step-back 25d-1. This causes a first feedback sound indicative of a start of medicament delivery because the plunger rod 17 has yet to move, or is in the processes of moving, from the first axial position towards the second axial position.

In FIG. 8C the cam surface 17b of the plunger rod 17 has interacted with the guide surface 23c-1 of the ring member 23-1 as the plunger rod 17 moved towards the second axial position. The ring member 23 was thus rotated in the second direction which is opposite to the first direction, as shown by the arrow. In this process, the feedback structure 23a-1 first moves under the second step 25f-1 causing it to be biased radially inwards a second time. This is followed by the feedback structure 23a-1 moving past the feedback structure 23a-1 and aligning with the second step-back 25. The feedback structure 23a-1 is thus relaxed and hits a radially inwards facing surface of the second step-back 25g-1. This causes a second feedback sound indicative of an end of medicament delivery.

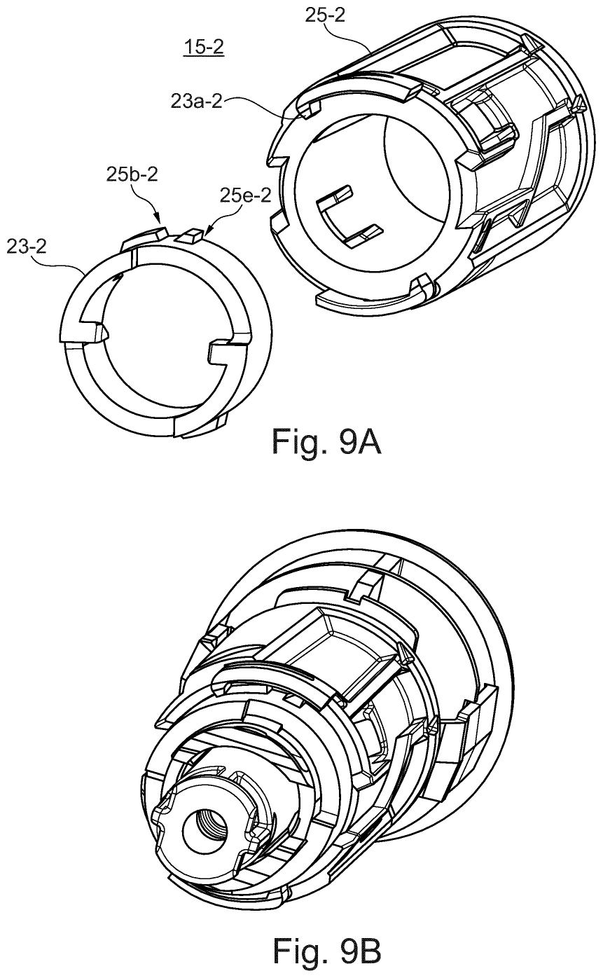

In another example of the feedback mechanism 15-2, the feedback structure and the first and second counter feedback structures could alternatively change place with each other, with the feedback structure 23a-2 being provided on the rotator 25-2 and the first and second counter feedback structures 25b-2 and 25e-2 being provided on the ring member 23-2, as shown in FIGS. 9A and 9B with the latter figure showing a state before medicament delivery has started.

The inventive concept has mainly been described above with reference to a few examples. However, as is readily appreciated by a person skilled in the art, other embodiments than the ones disclosed above are equally possible within the scope of the inventive concept, as defined by the appended claims.

1-13. (canceled)

14. A feedback mechanism for a medicament delivery device, comprising:
a rotator; and
a ring member arranged coaxially with the rotator,
wherein the rotator and the ring member are configured to be rotatable relative to each other,
wherein one of the rotator and the ring member has a radially resilient feedback structure and the other one of the rotator and the ring member has a first counter feedback structure configured to interact with the feedback structure when one of the rotator and the ring member is rotated relative to the other one of the rotator and the ring member,
wherein when the feedback structure interacts with the first counter feedback structure the feedback structure is configured to relax and hit the first counter feedback structure to thereby provide a first feedback sound.

15. The feedback mechanism of claim 14, wherein the feedback structure is an arm.

16. The feedback mechanism of claim 14, wherein the first counter feedback structure comprises a radial first step which when interacting with the feedback structure causes the feedback structure to become radially biased, and a radial first step-back following the first step in the circumferential direction of the feedback mechanism configured to be hit by the feedback structure when the feedback structure is relaxed to provide the first feedback sound.

17. The feedback mechanism of claim 16, wherein initial rotation of the rotator relative to the ring member causes the feedback structure to become radially biased by the first step.

18. The feedback mechanism of claim 17, wherein further relative rotation between the rotator and the ring member causes the feedback structure to relax by the first step-back.

19. The feedback mechanism of claim 14, wherein the rotator is configured to be rotated in a first direction and the ring member is configured to be rotated in a second direction opposite to the first direction.

20. The feedback mechanism of claim 17, wherein the rotator is configured to be rotated first causing interaction of the feedback structure with the first step, and the ring member is configured to be rotated second causing interaction of the feedback structure with the first step-back.

21. The feedback mechanism of claim 16, wherein the one of the rotator and the ring member which has the first counter feedback structure has a second counter feedback structure configured to interact with the feedback structure when the ring member is rotated relative to the rotator, wherein the second counter feedback structure comprises a radial second step following the first step-back in the circumferential direction, which when interacting with the feedback structure causes the feedback structure to become radially biased a second time, and a radial second step-back following the second step in the circumferential direction configured to be hit by the feedback structure when the feedback structure is relaxed a second time to provide a second feedback sound indicative of an end of medicament delivery.

22. The feedback mechanism as claimed in claim 21, wherein the rotator is configured to be rotated first causing interaction of the feedback structure with the first step and first step-back, and the ring member is configured to be rotated second causing interaction of the feedback structure with the second step and the second step-back.

23. The feedback mechanism of claim 14, comprising a plunger rod, wherein the rotator and the ring member are arranged around the plunger rod, the plunger rod being configured to move linearly from a first axial position to a second axial position relative to the rotator and the ring member, wherein the plunger rod has a cam surface and the ring member has a guide surface, the cam surface being configured to interact with the guide surface to cause rotation of the ring member relative to the rotator when the plunger rod is moved from the first axial position towards the second axial position, thereby causing the feedback structure to rotate relative to the first counter feedback structure such that the feedback structure is relaxed.

24. The feedback mechanism of claim 14, wherein the first feedback sound is a first click indicative of a start of medicament delivery or a second click indicative of an end of medicament delivery.

25. A medicament delivery device comprising a feedback mechanism of claim 14.

26. The medicament delivery device of claim 25, comprising:
a housing having a proximal end and a distal end;
a plunger rod biased towards the proximal end, arranged in the housing;
a rear cap comprising a radially flexible arm, wherein the rotator is configured to bear against the arm such that the arm engages with and holds the plunger rod in a first axial position; and
a delivery member cover configured to be moved linearly in the housing from an extended position to a retracted position relative to the housing, causing rotation of the rotator which enables the arm to disengage from the plunger rod to thereby release the plunger rod from the first position.
